(19) United States
(12) Patent Application Publication
(10) Pub. No.: US 2026/0132423 A1
(43) Pub. Date: May 14, 2026

(54) Method for Editing Bovine Gene Based on Pro-iCHI

(71) Applicant: Inner Mongolia University, Huhhot (CN)

(72) Inventors: Lei YANG, Huhhot (CN); Anqi DI, Huhhot (CN); Lishuang SONG, Huhhot (CN); Xuefei LIU, Huhhot (CN); Di WU, Huhhot (CN); Song WANG, Huhhot (CN); Zhenting HAO, Huhho (CN); Lige BU, Huhhot (CN); Chunling BAI, Huhhot (CN); Guanghua SU, Huhhot (CN); Zhuying WEI, Huhhot (CN); Li ZHANG, ZHANG (CN); Zhonghua LIU, Huhhot (CN); Shaorong GAO, Huhhot (CN); Guangpeng LI, Huhhot (CN)

(21) Appl. No.: 19/275,963

(22) Filed: Jul. 21, 2025

(30) Foreign Application Priority Data
Nov. 14, 2024 (CN) 202411620354.6

(51) Int. Cl. C12N 15/873; A61D 19/04; C12N 5/073; C12N 9/12; C12N 9/22; C12N 15/11; C12N 15/90

(52) U.S. Cl. CPC C12N 15/873; A61D 19/04; C12N 5/0604; C12N 9/1276; C12N 9/226; C12N 15/11; C12N 15/907; C12Y 207/07049; C07K 2319/00; C12N 2310/20; C12N 2501/727; C12N 2501/999; C12N 2510/00

(57) ABSTRACT

The present invention belongs to the field of molecular biology and genetics, and in particular relates to a method for editing a bovine gene based on Pro-iCHI. The present invention provides a method for editing a bovine gene based on Pro-iCHI. Protamine is transiently expressed in gene-edited b-haSCs, which are then injected to mature oocytes to obtain reconstructed embryos. Protamine can eliminate abnormal DNA methylation resulting from oocyte intracytoplasmic haSCs injection and enable the nucli to compress into sperm-like structures, and the obtained bovine Pro-iCHI embryos can successfully develop into blastocysts, with a blastocyst rate comparable to that of the embryos obtained by in vitro fertilization. Moreover, in the present invention, a protamine-encoding gene is inserted into a Saccharomyces cerevisiae protein expression vector for transient expression, which ensures that abnormal DNA methylation is erased, without integration into the genome resulting in the insertion of exogenous genes.

### CROSS REFERENCE TO RELATED APPLICATION

The present application claims the right of priority for Chinese patent application No. CN202411620354.6, filed with the China National Intellectual Property Administration on Nov. 14, 2024 and entitled “METHOD FOR EDITING BOVINE GENE BASED ON Pro-iCHI”, which is incorporated herein by reference in its entirety.

### REFERENCE TO SEQUENCE LISTING

A computer readable XML file entitled “GWP20241208216”, that was created on May 21, 2025, with a file size of about 14,965 bytes, contains the sequence listing for this application, has been filed with this application, and is hereby incorporated by reference in its entirety.

### TECHNICAL FIELD

The present invention belongs to the field of molecular biology and genetics, and in particular relates to a method for editing a bovine gene based on Protein and eoocyte intracytoplasmic haSCs injection (Pro-iCHI).

### BACKGROUND

Haploid cells contain only one set of chromosomes, so all genetic mutations in haploid cells are dominant mutations, which can be applied to gene screening at the mammalian cell level. In natural, the haploid cells in mammals are only oocyte and sperm. An oocyte is combined with sperm to form a diploid zygote, which eventually develops into a complete individual. Haploid embryos of mice, cattle and sheep can be obtained by methods such as parthenogenetic activation and microsurgical removal of the female pronucleus. Haploid embryonic stem cells (haSCs) are derived from haploid embryos. During in vitro culture, these haploid stem cells tend to undergo spontaneous diploidization. Therefore, flow cytometry is required to isolate and purify the haploid population, resulting in haploid embryonic stem cells that can be maintained long-term in vitro and used for biological research.

Studies have confirmed that gene-edited semi-cloned mice can be prepared from gene-edited sperm-like haploid cells by oocyte intracytoplasmic haSCs injection (iCHI) in mice. However, there are still some difficulties in applying this technique to bovine breeding. During the development of the reconstructed embryos, there are abnormal methylation modifications, which make it very difficult for the reconstructed embryos to develop into blastocysts, rendering them unable to be used for subsequent preparation of genome-edited animals.

### SUMMARY

The objective of the present invention is to remedy the deficiencies of the prior art. Gene editing is performed on cattle to erase abnormal methylation modifications resulting from iCHI, thereby obtaining iCHI blastocysts, which can successfully develop into embryos, similar to the blastocysts obtained by in vitro fertilization (IVF), so as to prepare the gene-edited cattle.

The present invention provides a method for editing a bovine gene based on Pro-iCHI, which method includes the following steps:
performing gene editing on bovine haploid androgenetic stem cells (b-haSCs) to obtain gene-edited b-haSCs;
transiently expressing protamine in the gene-edited b-haECs to obtain gene-edited Pro-b-haECs;
injecting the gene-edited Pro-b-haECs into mature oocytes to obtain reconstructed embryos (pro-iCHI);
activating the reconstructed embryos with ionomycin, and then incubating a resulting reconstructed embryos with 6-dimethylaminopurine to obtain bovine Pro-iCHI embryos.

In some embodiments, a method for the transiently expressing protamine in the gene-edited b-haSCs includes: transfecting the gene-edited b-haSCs with a recombinant vector;
the recombinant vector includes a Saccharomyces cerevisiae protein expression vector and a protamine-encoding gene inserted into the Saccharomyces cerevisiae protein expression vector.

In some embodiments, a mode for the transfection includes electro transfection.

In some embodiments, a concentration of the ionomycin is 5 μM to 10 μM; the activating is conducted for 5 min to 10 min.

In some embodiments, a concentration of the 6-dimethylaminopurine is 2 mM to 4 mM; the incubating is conducted for 5 h to 6 h.

In some embodiments, a mode for the performing gene editing on bovine haploid androgenetic stem cells includes introducing a gene editing system into bovine haploid androgenetic stem cells;
the gene editing system includes a base vector, and a prime editor and a fusion protein introduced into the base vector; the prime editor includes a guide RNA, a primer sequence, and a transcription template sequence for a to-be-edited gene; the guide RNA includes a spacer sequence for the to-be-edited gene;
the fusion protein includes a Cas9 protein in an H840A-nicked form (Cas9 (H840A)) and reverse transcriptase.

In some embodiments, the performing gene editing on bovine haploid androgenetic stem cells includes: performing gene editing on an MSTN gene of bovine haploid androgenetic stem cells.

In some embodiments, the nucleotide sequence of a spacer sequence used to locate the MSTN gene is set forth in SEQ ID NO: 4.

The present invention further provides the use of the method described in the above technical solution in the preparation of gene-edited cattle.

In some embodiments, the use includes: culturing the obtained bovine Pro-iCHI embryos, obtaining gene-edited blastocysts, then performing embryo transplantation to obtain gene-edited cattle.

### Beneficial Effects

The present invention provides a method for editing a bovine gene based on Pro-iCHI, including the following steps: performing gene editing on bovine androgenetic haploid embryonic stem cells to obtain gene-edited b-haSCs; transiently expressing protamine in the gene-edited b-haSCs, then injecting a resulting product into mature oocytes to obtain reconstructed embryos; activating the reconstructed embryos with ionomycin, incubating the reconstructed embryos with 6-dimethylaminopurine to obtain bovine Pro-iCHI embryos. In the present invention, protamine is transiently expressed, which can eliminate abnormal DNA methylation in the reconstructed embryos resulting from iCHI, and specifically eliminate abnormal modifications of Histone H3 trimethylated at lysine 4, Histone H3 trimethylated at lysine 9 and Histone H3 trimethylated at lysine 27 in the iCHI embryos, and enable the nuclei to compress into sperm-like structures. The results of the examples show that the bovine Pro-iCHI embryos obtained after the transient expression of protamine successfully develop into blastocysts, with a blastocyst rate comparable to that of the embryos obtained by in vitro fertilization.

Further, in the present invention, a protamine-encoding gene fragment is inserted into a protamine expression vector and then transiently expressed in the gene-edited b-haSCs, which ensures that protamine only functions to erase abnormal DNA methylation, and does not integrate into the genome resulting in the insertion of exogenous genes.

### BRIEF DESCRIPTION OF THE DRAWINGS

To more clearly illustrate the technical solutions in the examples of the present invention or in the prior art, the drawings required for the examples will be briefly described below.

FIG. 1 is a flow chart of the preparation of bovine iCHI embryos;

FIG. 2 is a graph showing the results of immunofluorescence of bovine iCHI embryos prepared in Comparative Example 1, in which the scale bar is 100 μm;

FIG. 3 is a statistical graph of histone modifications of reconstructed embryos obtained by different treatment modes;

FIG. 4 is a graph showing blastocyst images of pre-implantation embryos obtained by different treatment modes, in which the scale bar is 100 μm;

FIG. 5 is a line graph of the developmental rates of embryos obtained by different treatment modes;

FIG. 6 is a graph showing the results of immunostaining of H3K4me3, H3K9me3 and H3K27me3 in Test Example 1, in which the scale bar is 20 μm;

FIG. 7 is a schematic diagram of editing of the MSTN gene by the ePE system;

FIG. 8 is a graph showing the results of Sanger sequencing of the MSTN gene targeting site of b-haSCs;

FIG. 9 is a graph showing the results of Western blot analysis of MSTN proteins in wild-type b-haSCs and b-haSCs with MSTN edited;

FIG. 10 is a graph showing representative pictures of wild-type live cattle and MSTN′-live cattle;

FIG. 11 is a graph showing the results of Western blot analysis of MSTN proteins in wild-type live cattle and MSTN′-live cattle;

FIG. 12 is a graph showing the results of Sanger sequencing of targeting sites in wild-type live cattle and MSTN-live cattle.

### DETAILED DESCRIPTION OF THE EMBODIMENTS

The present invention provides a method for editing a bovine gene based on Pro-iCHI, including the following steps:
performing gene editing on bovine androgenetic haploid embryonic stem cells to obtain gene-edited b-haSCs;
transiently expressing protamine in the gene-edited b-haSCs to obtain gene-edited Pro-b-haSCs;
injecting the gene-edited Pro-b-haSCs into mature oocytes to obtain reconstructed embryos;
activating the reconstructed embryos with ionomycin, and then incubating the reconstructed embryos with 6-dimethylaminopurine to obtain bovine Pro-iCHI embryos.

As one embodiment, bovine androgenetic embryos are prepared in the present invention. As one embodiment, a method for preparing the bovine androgenetic embryos of the present invention may be injecting or removing. As one embodiment, steps of the injecting of the present invention include: removing the spindle of mature oocytes, and then injecting sperm into the mature oocytes to obtain reconstructed embryos; activating the reconstructed embryos with ionomycin, then incubating the reconstructed embryos with 6-dimethylaminopurine in a G1 culture medium for 3 days, and then transferring the reconstructed embryos to a G2 culture medium for 4.5 days to obtain bovine androgenetic embryos. As one embodiment, steps of the removing of the present invention include: preparing bovine zygote by means of in vitro fertilization, removing the female pronuclei of the bovine zygote, then culturing resulting bovine zygote in a G1 culture medium for 3 days, and then transferring resulting bovine zygote to a G2 culture medium for 4.5 days to obtain bovine androgenetic embryos.

After obtaining the bovine androgenetic embryos, as one embodiment, in the present invention, the zona pellucida of the bovine androgenetic embryos are removed, and resulting bovine androgenetic embryos are cultured to obtain bovine haploid androgenetic stem cells. As one embodiment, the proportion of haploid cells is maintained by sorting using a flow cytometer per 5 passages of culture.

After obtaining the bovine haploid androgenetic stem cells, in the present invention, gene editing is performed on the bovine haploid androgenetic stem cells to obtain gene-edited b-haESCs. As one embodiment, a mode for the performing gene editing on bovine haploid androgenetic stem cells includes introducing a gene editing system into bovine haploid androgenetic stem cells; as one embodiment, the gene editing system includes a base vector, and a prime editor and a fusion protein introduced into the base vector; the prime editor includes a guide RNA, a primer sequence, and a transcription template sequence for a to-be-edited gene; the guide RNA includes a spacer sequence for the to-be-edited gene; the fusion protein includes a Cas9 protein in an H840A-nicked form and reverse transcriptase; the guide RNA for the to-be-edited gene guides a Cas9 protein in an H840A-nicked form to cleave single-stranded DNA of the to-be-edited gene, the primer sequence recognizes a complementary sequence before the cleavage site, the reverse transcriptase performs reverse transcription using the transcription template sequence as a template and polymerizes the transcription template sequence onto the cleaved DNA strand. As one embodiment, the performing gene editing on bovine androgenetic haploid embryonic stem cells includes: performing gene editing on an MSTN gene of bovine haploid androgenetic stem cells. As one embodiment, the nucleotide sequence of a spacer sequence used to locate the MSTN gene is set forth in SEQ ID NO: 4; as one embodiment, the nucleotide sequence of a prime editor used to locate the MSTN gene is set forth in SEQ ID NO: 3. In the embodiments of the present invention, the MSTN gene is taken as an example for illustration and cannot be merely construed as the full scope of protection of the present invention.

After obtaining gene-edited b-haSCs, in the present invention, protamine is transiently expressed in the gene-edited b-haSCs to obtain gene-edited Pro-b-haSCs. As one embodiment, a method for the transiently expressing protamine in the gene-edited b-haSCs includes: transfecting the gene-edited b-haSCs with a recombinant vector; the recombinant vector includes a Saccharomyces cerevisiae protein expression vector and a protamine-encoding gene inserted into the Saccharomyces cerevisiae protein expression vector. As one embodiment, a mode for the transfection includes electro transfection. In the present invention, a protamine-encoding gene fragment is inserted into a non-integrated expression vector and then transiently expressed in the gene-edited b-haSCs, which ensures that protamine only functions to erase abnormal DNA methylation, and does not integrate into the genome resulting in the insertion of exogenous genes.

After obtaining gene-edited Pro-b-haSCs, in the present invention, the gene-edited Pro-b-haSCs are injected into mature oocytes to obtain reconstructed embryos. As one embodiment, the ratio of the number of Pro-b-haSCs and mature oocytes is 1:1.

After obtaining the reconstructed embryos, in the present invention, after activating the reconstructed embryos with ionomycin, the reconstructed embryos are incubated with 6-dimethylaminopurine to obtain bovine Pro-iCHI embryos. As one embodiment, a concentration of the ionomycin is 5 μM to 10 μM; as another embodiment, a concentration of the ionomycin is 5 μM. As one embodiment, a time for the activation is 5 min to 10 min; as another embodiment, a time for the activation is 5 min. As one embodiment, a concentration of the 6-dimethylaminopurine is 2 mM to 4 mM; as another embodiment, a concentration of the 6-dimethylaminopurine is 2 mM; As one embodiment, a time for the incubation is 5 h to 6 h; as another embodiment, a time for the incubation is 5 h.

The present invention further provides the use of the method described in the above technical solution in the preparation of gene-edited cattle. As one embodiment, the use includes: culturing the bovine Pro-iCHI embryos obtained by the method described in the above technical solution to obtain gene-edited blastocysts, and then performing embryo transplantation to obtain gene-edited cattle. As one embodiment, the culture includes a first culture and a second culture; a culture medium used for the first culture is a G1 culture medium and a culture time for the first culture is 72 h; a culture medium used for the second culture is a G2 culture medium.

In the present invention, protamine is transiently expressed in gene-edited b-haSCs, which can eliminate abnormal DNA methylation in the embryos resulting from oocyte intracytoplasmic haSCs injection, and specifically eliminate abnormal modifications of Histone H3 trimethylated at lysine 4, Histone H3 trimethylated at lysine 9 and Histone H3 trimethylated at lysine 27 in the iCHI embryos, and enable the nuclei to compress into sperm-like structures. The results of the examples show that the bovine Pro-iCHI embryos obtained after the transient expression of protamine successfully develop into blastocysts, with a blastocyst rate comparable to that of the embryos obtained by in vitro fertilization.

To further illustrate the present invention, the method for editing a bovine gene based on Pro-iCHI provided by the present invention is described in detail below in conjunction with the drawings and examples, which cannot be construed as limiting the scope of protection of the present invention, however.

The composition and source of the reagents used in the examples of the present invention are as follows:
Hoechst 33342 staining solution: purchased from Sigma;
M2 in-vitro operating solution: purchased from Beijing Yihe Technology Co., Ltd.;
G1 culture medium: purchased from Vitrolife;
G2 culture medium: purchased from Vitrolife;
mTeSR1 culture medium: purchased from Stemcell;
KSOM-AA culture medium: purchased from Beijing Yihe Technology Co., Ltd.

### Example 1

### 1. Preparation of Bovine Androgenetic Embryos—Injecting

After bovine ovaries obtained from the slaughterhouse were brought back to the laboratory, oocytes were obtained by suction. After the oocytes were subjected to in-vitro maturation, the oocytes with the first polar body extruded were obtained, which were then stained with 5 μg/mL Hoechst 33342 staining solution for 10 min and briefly irradiated under UV light for 1-2 s to determine the position of the spindle. Using a Nikon inverted microscope equipped with a 37° C. hot stage, the spindle was removed in the M2 in-vitro operating solution containing 5 μg/mL cytochalasin B (CB), and one sperm was inserted into the cytoplasm. Subsequently, the reconstructed embryos were activated in 5 μM ionomycin solution for 5 min, then placed in 2 mM 6-dimethylaminopurine, and incubated under the conditions of 38.5° C., 5% CO₂ for 5 h. After that, resulting reconstructed embryos were cultured in the G1 culture medium for 3 days, then transferred to the G2 culture medium, and cultured for 4.5 days to obtain bovine androgenetic embryos.

### 2. Preparation of Bovine Androgenetic Embryos—Removing

After bovine ovaries obtained from the slaughterhouse were brought back to the laboratory, oocytes were obtained by suction. After the oocytes were subjected to in vitro maturation, the oocytes with the first polar body extruded were obtained. The mature oocytes with the first polar body extruded were subjected to in vitro fertilization to obtain zygotes. The zygotes were stained with 5 μg/mL Hoechst 33342 staining solution for 10 min in the G1 culture medium and briefly irradiated under UV light for 1-2 s to determine the location of the male and female pronuclei. The female pronucleus was then removed by micromanipulation. Subsequently, the zygotes were incubated in the G1 culture medium for 3 days, then transferred to the G2 culture medium, and cultured under the conditions of 38.5° C., 5% CO₂, and saturated humidity for about 4.5 days to obtain bovine androgenetic embryos.

### 3. Culture of Bovine Androgenetic Haploid Embryonic Stem Cells

(1) Since the blastocyst rates of the bovine androgenetic embryos obtained by the two methods in step 1 and step 2 were similar, being 31.16% and 30.21%, respectively, the above two methods could be used for the preparation and culture of bovine haploid androgenetic stem cells.

In this step, the bovine androgenetic blastocysts obtained in step 1 were treated with 0.25 wt. % pronase to remove the zona pellucida, and subsequently placed in the FACE culture medium and cultured until stem cell colonies grew. For the FACE culture medium, the mTeSR1 culture medium was used as a basal culture medium, and 2.5 μM IWR1-1-endo, 10 ng/ml fibroblast growth factor 2 (FGF2), 3 μM CHIR99021, and 10 ng/ml activin A were added to every 500 mL of the mTeSR1 culture medium.

(2) Since bovine haploid androgenetic stem cells (b-haSCs) may undergo spontaneous diploidization during the culture, to maintain the proportion of haploid cells, b-haSCs were continuously sorted by flow cytometry during the culture in step 3. The specific procedures were as follows: At 37° C., the stem cells after 5 passages of culture were incubated with 10 μg/mL Hoechst 33342 staining solution for 30 min. The cells were gently mixed every 10 min to ensure uniform staining. The cell population of haploid embryonic stem cells was selected and collected, and spontaneous diploid stem cells were removed. The obtained stem cells were purified. The results showed that the proportion of b-haSCs could reach 20% after the initial sorting, and as the number of sortings increased, the proportion could eventually reach 80%.

### Example 2

### 1. Construction of Episomal-Prm1 Vector

(1) Using the #138320 vector (pCW57-MCS1-P2A-MCS2-mDux) purchased from the addgene website as a backbone, double enzymatic cleavage was performed with Nhe I and Mlu I to obtain two fragments of 2184 bp and 7847 bp in size, and the fragment of 7847 bp in size was recovered by the agarose gel DNA extraction kit (Tiangen). The mouse-derived Prm1 gene and enhanced green fluorescent protein (EGFP) gene, both with Nhe I and Mlu I at their two ends, were synthesized by Sangon Biotech (Shanghai). The two fragments were ligated together by a DNA ligase to form a Prm1 plasmid. The Prm1 plasmid was subjected to single enzymatic cleavage at the Not I enzymatic cleavage site to obtain an enzymatic cleavage fragment.

(2) Using the #84031 vector (pCRISPR-S12) purchased from the addgene website as a backbone, PCR amplification was performed using PCR amplification primers (episomal-F: 5′-ACCACCGCACAGCAAAACGGGTAGCATATGCTTC-3′, SEQ ID NO: 1; episomal-R: 5′-CAGGTCTGAAGATCAATGTCTGACGAGGGGCCAG-3′, SEQ ID NO: 2) to obtain a PCR product of 4047 bp in size.

(3) The enzymatic cleavage fragment obtained in step (1) and the PCR product obtained in step (2) were subjected to homologous recombination by the one-step cloning kit (purchased from Nanjing Vazyme Biotech Co., Ltd.) to finally obtain the episomal-prm1 vector. The episomal vector element in this vector allows protamine to function without integrating into the genome.

2. The b-haSCs obtained in Example 1 were transfected with the episomal-prm1 vector obtained in step 1 by the electroporation method, and protamine (Pro) was transiently expressed in the b-haSCs to obtain Pro-b-haSCs.

### Comparative Example 1

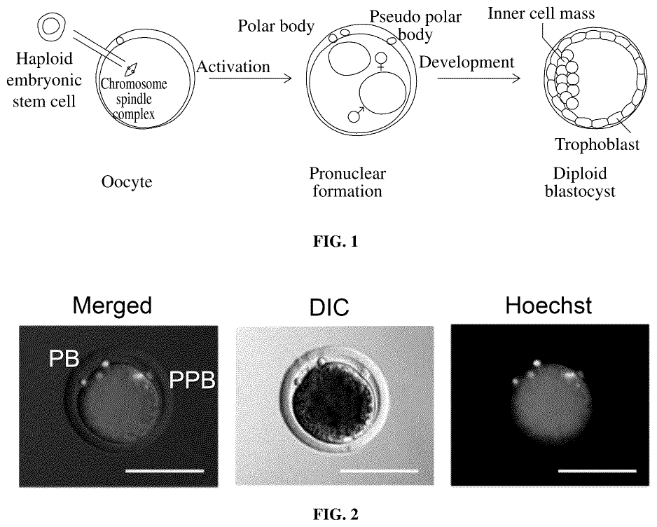

Bovine oocytes were prepared by referring to the steps of Example 1. By oocyte intracytoplasmic haSCs injection (iCHI), the b-haSCs obtained in Example 1 were injected, instead of sperm, into mature bovine oocytes according to the flow shown in FIG. 1 to prepare bovine iCHI embryos. The specific procedures were as follows:

Using a Nikon inverted microscope equipped with a 37° C. hot stage, one b-haSC obtained in Example 1 was injected into mature bovine oocytes in the M2 in-vitro operating solution containing 5 μg/mL cytochalasin B. Subsequently, the reconstructed embryo was activated in 5 μM ionomycin solution for 5 min, then placed in 2 mM 6-dimethylaminopurine, and incubated under the conditions of 38.5° C., 5% CO₂, and saturated humidity for 5 h to obtain a bovine iCHI embryo. The entire operation process was performed under a Nikon inverted microscope equipped with a 37° C. hot stage and a manipulator arm, and the formation of pronucleus and polar body was observed. The reconstructed embryo is as shown in FIG. 2, in which PB represents a polar body; PPB represents a pseudo polar body; scale bar, 100 μm.

### Comparative Example 2

### Preparation of In Vitro Fertilization (IVF) Embryos

The oocytes obtained from the ovaries were subjected to in-vitro maturation and then placed in the fertilization solution, and added with sperm at a sperm-to-oocyte ratio of 500:1. After 6 hours of fertilization, the sperm were washed away. The zygotes were placed in the G1 culture medium and cultured for 3 days, and then transferred to the G2 culture medium and cultured for 4 days until the blastocyst stage.

### Example 3

By referring to the steps of Comparative Example 1, the Pro-b-haSCs obtained in Example 2 were injected, instead of sperm, into mature bovine oocytes to prepare bovine Pro-iCHI embryos.

### Test Example 1

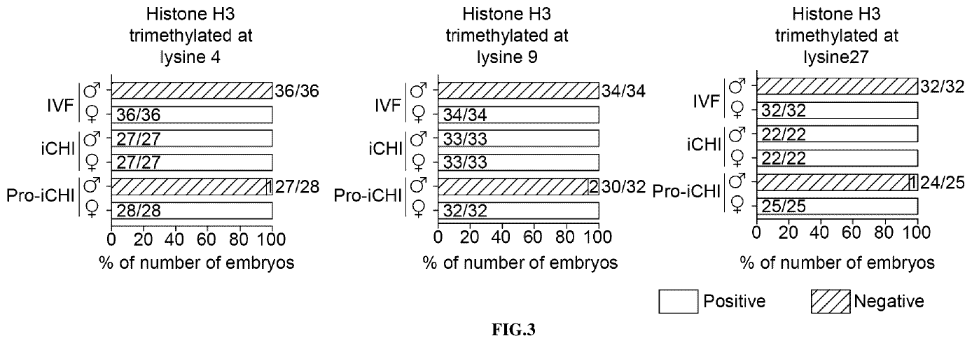

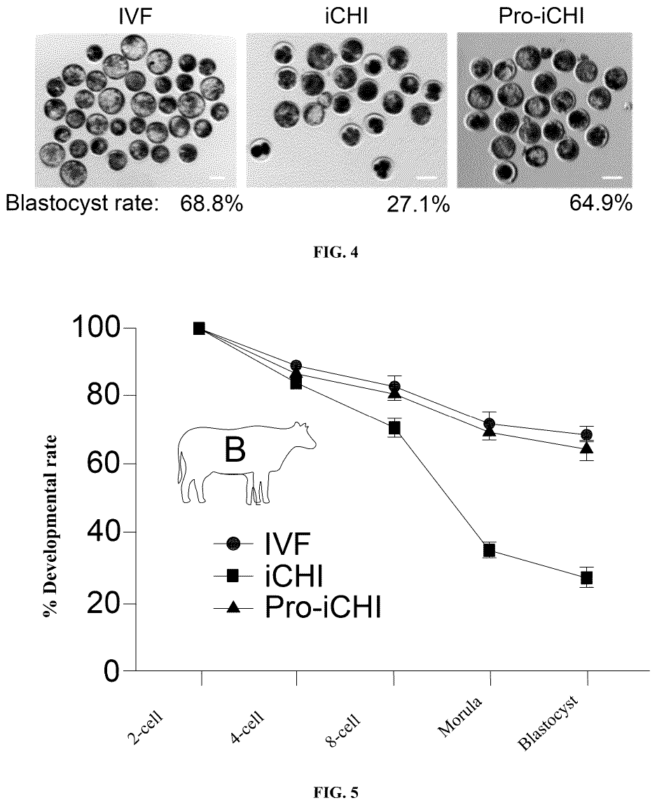

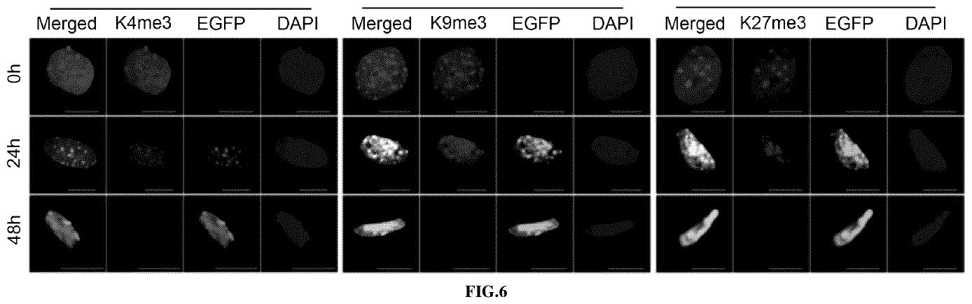

The development conditions of the bovine iCHI embryos obtained in Comparative Example 1, the IVF embryos obtained in Comparative Example 2, and the bovine Pro-iCHI embryos obtained in Example 3 were each statistically analyzed, and the proportions of cells in different periods of time were counted. The results are shown in FIG. 3 to FIG. 5 and Table 1.

TABLE 1
Development conditions of different embryos

| | | | Cleavage | Cleavage | Cleavage | | |
|---|---|---|---|---|---|---|---|
| | | Number | rate of 2- | rate of 4- | rate of 8- | | |
| | | of 1-cell | cell | cell | cell | | Blastocyst |
| | Number of | embryos | embryos | embryos | embryos | Morula | rate |
| Treatment | repetitions | (pieces) | (%) | (%) | (%) | rate (%) | (%) |
| Comparative | 3 | 154 | 89.59 ± 1.87 | 89.12 ± 1.34 | 83.28 ± 2.63 | 72.04 ± 3.30 | 69.02 ± 1.73 |
| Example 2 | | | | | | | |
| (IVF) | | | | | | | |
| Comparative | 3 | 321 | 87.31 ± 0.90 | 84.21 ± 1.61 | 70.98 ± 2.40*** | 35.10 ± 1.37*** | 27.12 ± 2.42*** |
| Example 1 | | | | | | | |
| (iCHI) | | | | | | | |
| Example 3 | 3 | 314 | 89.26 ± 1.12 | 86.73 ± 2.23 | 80.82 ± 1.64 | 69.72 ± 2.37 | 64.91 ± 3.3 |
| (Pro-iCHI) | | | | | | | |

Note:
*P < 0.05, **P < 0.01, ***P < 0.001, two-tailed t-test was employed; Cleavage rate of 2-cell embryos = (number of 2-cell embryos/number of cell embryos) × 100%; Cleavage rate in the remaining cleavage stages = (number of cleaved embryos/number of cleaved 2-cell embryos) × 100%.

As can be seen from FIG. 3 to FIG. 5 and Table 1, the blastocyst rate of the bovine iCHI embryos obtained in Comparative Example 1 was significantly lower than that of the IVF embryos, and there were abnormalities in the embryonic development stage, resulting in failure to develop into blastocysts. Abnormal methylation modification is the major cause of impairment in embryonic development, and mainly includes abnormalities of Histone H3 trimethylated at lysine 4 (H3K4me3), Histone H3 trimethylated at lysine 9 (H3K9me3), and Histone H3 trimethylated at lysine 27 (H3K27me3). However, the developmental rate of the Pro-iCHI embryos was similar to that of the IVF embryos. This indicates that transiently expressed Prm1 can effectively erase abnormal methylation modifications, enabling Pro-iCHI to restore to the level of IVF. The blastocyst rate of bovine Pro-iCHI embryos obtained in Example 3 was comparable to that of the embryos obtained by in vitro fertilization (IVF) in Comparative Example 2. In the present invention, by improving the iCHI technology, Pro-iCHI embryos applied to cattle were obtained, ensuring the feasibility of subsequent preparation of genome-edited animals.

Immunofluorescence verification was performed on b-haSCs transiently expressing Prm1. The results are shown in FIG. 6. As can be seen from FIG. 6, transient expression of protamine in b-haSCs in a non-integrated manner can eliminate the abnormal modifications of H3K4me3, H3K9me3 and H3K27me3 in the bovine iCHI embryos and enable the nuclei to compress into sperm-like structures.

### Example 4

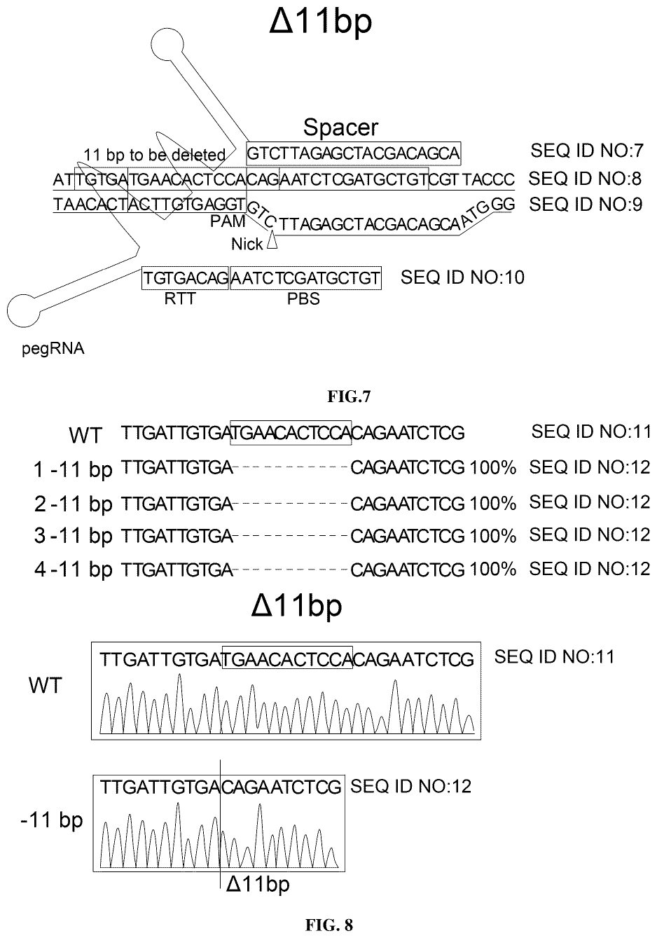

(1) Myostatin (MSTN), a member of the transforming growth factor β (TGFβ) superfamily, is mainly expressed in skeletal muscle, secreted into the extracellular space, and transported through the blood circulation to various tissue organs to play a regulating role. Mutations in the MSTN gene will cause significant changes in the muscle phenotype of animals, resulting in the phenomenon of “double muscling”. Thus, in this example, the MSTN gene was selected as the subject of gene editing. Prime editors (PEs) were selected to edit the MSTN gene of the b-haSCs obtained in Example 1. The editing principle of the prime editors is shown in FIG. 7: a single-stranded DNA is cleaved by nCas9 nickase under the guidance of a spacer sequence on a guide RNA (pegRNA), a PBS sequence (primer sequence) recognizes a complementary sequence before the cleavage site, and reverse transcription is performed using an RTT sequence (transcription template sequence) as a template, so as to polymerize a sequence of interest onto the nicked DNA strand.

The prime editors used in this example consist of gRNA scaffold, RTT and PBS, and the specific nucleotide sequence is 5′-acgacagcatcgagattctggttttagagctagaaatagcaagttaaaataaggctagtccgttatcaacttgaaaaagtggcaccgagtcgg tgctgtgacagaatctcgatgctgt-3′ (SEQ ID NO: 3), denoted as bGHpoly-gRNA scaffold; where the spacer sequence in the gRNA scaffold is: 5′-acgacagcatcgagattctg-3′ (SEQ ID NO: 4); the sequence of the gRNA scaffold is: 5′-gttttagagctagaaatagcaagttaaaataaggctagtccgttatcaacttgaaaaagtggcaccgagtcggtgc-3′ (SEQ ID NO: 5); the sequence of RTT is: 5′-tgtgacag-3′; the sequence of PBS is: 5′-aatctcgatgctgt-3′ (SEQ ID NO: 6).

(2) Using the #132776 vector (pCMV-PE2-P2A-GFP) purchased from the addgene website as a backbone, double enzymatic cleavage was performed with SgrD I and Pme I, and a fragment of interest was obtained by DNA gel electrophoresis and recovered with the agarose gel DNA extraction kit (Tiangen) to obtain the recovered fragment; the bGHpoly-gRNA scaffold sequence fragment in step (1) was synthesized by Sangon Biotech (Shanghai). The two fragments were integrated using the ClonExpress Ultra One-Step Cloning Kit V2 (Vazyme). Using the #84031 vector (pCRISPR-S12) purchased from the addgene website as a backbone, double enzymatic cleavage was performed with BamH I and Pac I, and homologous recombination was performed with the integrated fragment from the previous step to obtain an episomal plasmid-based PE (ePE) plasmid of the non-integrating prime editor.

(4) The ePE obtained in step (3) was transfected into the b-haSCs obtained in Example 1 by the transfection experiment. After culture, DNA was extracted from the b-haSCs. and the gene editing condition of MSTN in the b-haSCs was detected by the dideoxy sequencing method (Sanger sequencing method). As can be seen from the results, an 11 bp deletion occurred in exon 2 of the MSTN locus (FIG. 8).

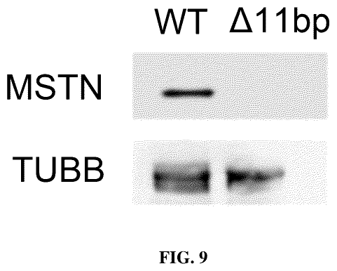

(5) After the transfection in step (4), the culture media of the wild-type b-haSCs (denoted as WT), and b-haSCs with an 11 bp deletion in exon 2 of the MSTN locus (denoted as Allbp) were discarded, and Tryple (Gibco) was added to digest cells for 3 min. The cell suspension was collected and centrifuged, and then the liquid was discarded. 1 mL of cell lysis buffer (volume ratio of phenylmethylsulfonyl fluoride:RIPA lysis buffer was 1:99) to the cell pellet to resuspend the cells. The cell suspension was left to stand at 4° C. for 20 min, placed in a 4° C. centrifuge, and centrifuged at 12,000 rmp for 20 min, and then the supernatant was pipetted to obtain the protein. With the TUBB protein as an internal reference, the gene editing condition of MSTN was verified at the protein level by Western Blot. The results indicated that the b-haSC also showed MSTN knockout at the protein level (FIG. 9).

### Example 5

(1) The episomal-prm1 vector obtained in Example 2 was transfected into the b-haSCs with MSTN knocked out obtained in Example 4 by the electroporation method, and protamine was transiently expressed in the b-haSCs with MSTN knocked out to obtain MSTN^{+/-} Pro-b-haSCs.

(2) By referring to the steps of Comparative Example 1, MSTN^{+/-} Pro-b-haSCs were injected, instead of sperm, into mature bovine oocytes to prepare MSTN^{+/-} bovine Pro-iCHI embryos.

### Test Example 2

The development conditions of the Pro-iCHI embryos (wild-type) prepared in Example 3 and the MSTN^{+/-} bovine Pro-iCHI embryos obtained in Example 5 were each statistically analyzed, and the proportions of cells in different periods of time were counted. The results are shown in Table 2.

TABLE 2
Development conditions of different embryos

| | | Number of | Number of 2-cell | Blastocyst |
|---|---|---|---|---|
| | Number of | reconstructed | embryos (rate of 2- | (number, |
| Treatment | repetitions | embryos | cell embryos) | blastocyst rate) |
| Example 3 (wild- | 3 | 51 | 45 (88.24%) | 29 (64.44%) |
| type) | | | | |
| Example 5 (MSTN^{+/-}) | 3 | 66 | 58 (87.88%) | 37 (63.79%) |

As can be seen from Table 2, gene editing of MSTN did not affect the blastocyst rate of the Pro-iCHI embryos, and there was no significant difference between the two.

### Example 6

(1) The bovine Pro-iCHI embryos (wild-type) obtained in Example 3 and the MSTN^{+/-} bovine Pro-iCHI embryos obtained in Example 5 were separately cultured in the G1 culture medium and the G2 culture medium for 7-8 days to obtain blastocysts for subsequent embryo transplantation. The specific procedures were as follows:

Using a Nikon inverted microscope equipped with a 37° C. hot stage, the bovine Pro-iCHI embryos (wild-type) obtained in Example 3 and the MSTN^{+/-} bovine Pro-iCHI embryos obtained in Example 5 were injected into mature bovine oocytes in the M2 in-vitro operating solution containing 5 μg/mL cytochalasin B (CB). Subsequently, the reconstructed embryos were placed in the KSOM-AA embryonic culture medium for 1 h and then activated by 5 μM ionomycin (Ionomycin, I) for 5 min. Subsequently, the reconstructed embryos were placed in 10 μg/mL cycloheximide (CHX) for 5 h, cultured in the G1 culture medium for 72 h, and then transferred to G2 and cultured until the blastocyst stage.

(2) The recipient cows were subjected to estrus treatment. Vaginal progesterone inserts (CIDR) were put into the recipient cows. On day 7, the vaginal progesterone inserts were removed, and prostaglandin (PG) was injected. Subsequently, the estrus conditions of the recipient cows were observed and recorded. After 7 days of estrus, the blastocysts obtained in step (1) were transplanted into the recipient cows. The blastocysts used for transplantation were stored in the M199 culture medium supplemented with 15% fetal calf serum. On day 35 after the transplantation, the pregnancy conditions were determined by a rectal ultrasound EVO scanner.

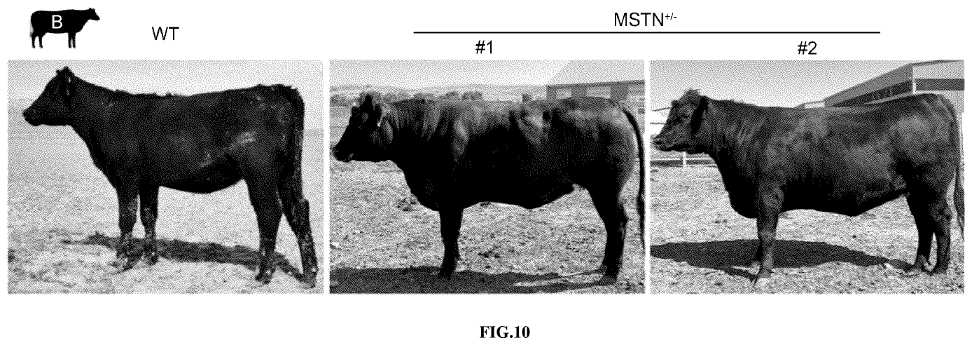

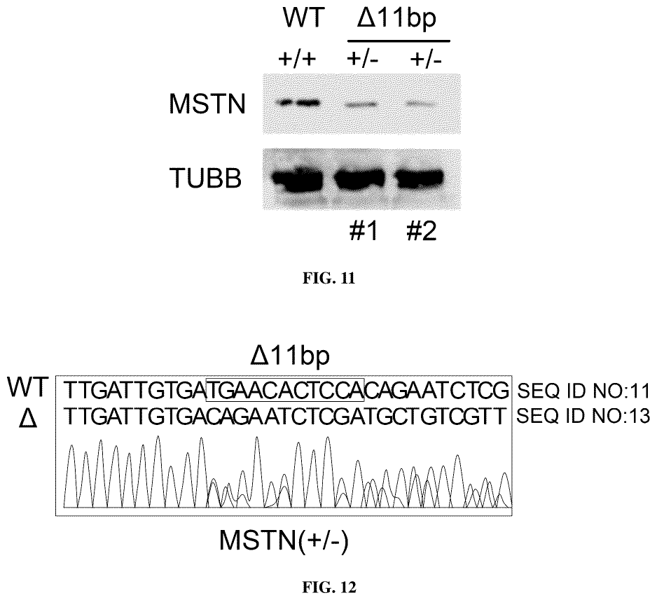

In this example, 15 recipient cows were subjected to the transplantation, with one embryo transplanted into each cow. After 287 days of pregnancy, two (13.3%, 2/15) MSTN^{+/-} calves with a “double muscling” phenotype were successfully born (FIG. 10 and Table 3). Live samples were collected from the born calves. The calves obtained by transplanting blastocysts with MSTN^{+/-}-knocked out were verified at the protein level by Western Blot as MSTN^{+/-}-gene-edited cattle and exhibited the double muscling phenotype. In addition, Sanger sequencing indicated that an 11 bp deletion occurred in the MSTN gene (FIG. 11 and FIG. 12).

TABLE 3
Transplantation and production conditions for different embryos

| | Number of embryos | Number of | Number of calves born (% |
|---|---|---|---|
| Treatment | transplanted per recipient | recipients | of transplanted embryos) |
| Example 1 (wild-type) | 1 | 7 | 1 (14.29) |
| Example 3 (MSTN^{+/-}) | 1 | 15 | 2 (13.33) |

As can be seen from the above content, the technical solutions provided by the present invention can eliminate abnormal DNA methylation resulting from oocyte intracytoplasmic haSCs injection and enable the nuclei to compress into sperm-like structures, and the obtained bovine Pro-iCHI embryos can successfully develop into blastocysts, with a blastocyst rate comparable to that of the embryos obtained by in vitro fertilization.

Although the examples described above have provided a detailed description of the present invention, they are only some, rather than all of the examples of the present invention. All other examples that can be obtained according to the examples of the present invention without involving any inventive effort shall fall within the scope of protection of the present invention.

What is claimed is:

1. A method for editing a bovine gene based on Pro-iCHI, comprising the following steps:
performing gene editing on bovine androgenetic haploid embryonic stem cells to obtain gene-edited b-haSCs;
transiently expressing protamine in the gene-edited b-haSCs to obtain gene-edited Pro-b-haSCs;
injecting the gene-edited Pro-b-haSCs into mature oocytes to obtain reconstructed embryos;
activating the reconstructed embryos with ionomycin, and then incubating a resulting reconstructed embryos with 6-dimethylaminopurine to obtain bovine Pro-iCHI embryos.

2. The method according to claim 1, wherein the transiently expressing protamine in the gene-edited b-haSCs is conducted as follows: transfecting the gene-edited b-haSCs with a recombinant vector;
the recombinant vector comprises a Saccharomyces cerevisiae protein expression vector and a protamine-encoding gene inserted into the Saccharomyces cerevisiae protein expression vector.

3. The method according to claim 2, wherein a mode for the transfection comprises electro transfection.

4. The method according to claim 1, wherein a concentration of the ionomycin is 5 μM to 10 μM; and the activating is conducted for 5 min to 10 min.

5. The method according to claim 1, wherein a concentration of the 6-dimethylaminopurine is 2 mM to 4 mM; and the incubating is conducted for 5 h to 6 h.

6. The method according to claim 1, wherein a mode for the performing gene editing on bovine androgenetic haploid embryonic stem cells comprises introducing a gene editing system into bovine androgenetic haploid embryonic stem cells;
the gene editing system comprises a base vector, and a prime editor and a fusion protein introduced into the base vector; the prime editor comprises a guide RNA, a primer sequence, and a transcription template sequence for a to-be-edited gene; and the guide RNA comprises a spacer sequence for the to-be-edited gene;
the fusion protein comprises a Cas9 protein in an H840A-nicked form and reverse transcriptase.

7. The method according to claim 1, wherein the gene editing is performed on an MSTN gene of bovine haploid androgenetic stem cells.

8. The method according to claim 7, wherein the nucleotide sequence of a spacer sequence used to locate the MSTN gene is set forth in SEQ ID NO: 4.

9. A method for preparing gene-edited cattle, comprising culturing the obtained bovine Pro-iCHI embryos in claim 1, obtaining gene-edited blastocysts, and then performing embryo transplantation to obtain gene-edited cattle.

10. The method according to claim 7, wherein the transiently expressing protamine in the gene-edited b-haSCs is conducted as follows: transfecting the gene-edited b-haSCs with a recombinant vector;
the recombinant vector comprises a Saccharomyces cerevisiae protein expression vector and a protamine-encoding gene inserted into the Saccharomyces cerevisiae protein expression vector.

11. The method according to claim 7, wherein a mode for the transfection comprises electro transfection.

12. The method according to claim 7, wherein a concentration of the ionomycin is 5 μM to 10 μM; and the activating is conducted for 5 min to 10 min.

13. The method according to claim 7, wherein a concentration of the 6-dimethylaminopurine is 2 mM to 4 mM; and the incubating is conducted for 5 h to 6 h.

14. The method according to claim 7, wherein a mode for the performing gene editing on bovine androgenetic haploid embryonic stem cells comprises introducing a gene editing system into bovine androgenetic haploid embryonic stem cells;
the gene editing system comprises a base vector, and a prime editor and a fusion protein introduced into the base vector; the prime editor comprises a guide RNA, a primer sequence, and a transcription template sequence for a to-be-edited gene; and the guide RNA comprises a spacer sequence for the to-be-edited gene;
the fusion protein comprises a Cas9 protein in an H840A-nicked form and reverse transcriptase.


SEQUENCE LISTING (13 sequences)

SEQ ID NO: 1 - DNA, 34 bp, synthetic construct
  source 1..34: mol_type = other DNA; note = Primer episomal-F
accaccgcacagcaaaacgggtagcatatgcttc

SEQ ID NO: 2 - DNA, 34 bp, synthetic construct
  source 1..34: mol_type = other DNA; note = Primer episomal-R
caggtctgaagatcaatgtctgacgaggggccag

SEQ ID NO: 3 - DNA, 118 bp, synthetic construct
  source 1..118: mol_type = other DNA; note = Specific nucleotide sequence of prime editors
acgacagcatcgagattctggttttagagctagaaatagcaagttaaaataaggctagtccgttatcaacttgaaaaagtggcaccgagtcggtgctgtgacagaatctcgatgctgt

SEQ ID NO: 4 - DNA, 20 bp, synthetic construct
  source 1..20: mol_type = other DNA; note = The spacer sequence in the gRNA scaffold
acgacagcatcgagattctg

SEQ ID NO: 5 - DNA, 76 bp, synthetic construct
  source 1..76: mol_type = other DNA; note = The sequence of the gRNA scaffold
gttttagagctagaaatagcaagttaaaataaggctagtccgttatcaacttgaaaaagtggcaccgagtcggtgc

SEQ ID NO: 6 - DNA, 14 bp, synthetic construct
  source 1..14: mol_type = other DNA; note = The sequence of PBS
aatctcgatgctgt

SEQ ID NO: 7 - RNA, 20 nt, synthetic construct
  source 1..20: mol_type = other RNA; note = Spacer sequence of pegRNA
acgacagcatcgagattctg

SEQ ID NO: 8 - DNA, 43 bp, synthetic construct
  source 1..43: mol_type = other DNA; note = Fragments of MSTN gene
attgtgatgaacactccacagaatctcgatgctgtcgttaccc

SEQ ID NO: 9 - DNA, 43 bp, synthetic construct
  source 1..43: mol_type = other DNA; note = Fragment of MSTN gene
gggtaacgacagcatcgagattctgtggagtgttcatcacaat

SEQ ID NO: 10 - RNA, 22 nt, synthetic construct
  source 1..22: mol_type = other RNA; note = RTT and PBS sequences of pegRNA
tgtgacagaatctcgatgctgt

SEQ ID NO: 11 - DNA, 31 bp, synthetic construct
  source 1..31: mol_type = other DNA; note = Fragment of WT
ttgattgtgatgaacactccacagaatctcg

SEQ ID NO: 12 - DNA, 20 bp, synthetic construct
  source 1..20: mol_type = other DNA; note = Fragment of edited MSTN gene
ttgattgtgacagaatctcg

SEQ ID NO: 13 - DNA, 31 bp, synthetic construct
  source 1..31: mol_type = other DNA; note = Fragment of edited MSTN gene
ttgattgtgacagaatctcgatgctgtcgtt